(19) United States
(12) Patent Application Publication
(10) Pub. No.: US 2011/0223156 A1
(43) Pub. Date: Sep. 15, 2011

(54) REVERSIBLE GEL PROTEIN FORMULATION

(76) Inventor: Andrei A. Raibekas, Thousand Oaks, CA (US)

(21) Appl. No.: 13/046,470

(22) Filed: Mar. 11, 2011

Related U.S. Application Data
(60) Provisional application No. 61/313,044, filed on Mar. 11, 2010.

(51) Int. Cl. A61K 39/395; A61K 38/02; A61K 38/43; A61K 38/18

(52) U.S. Cl. 424/133.1; 514/1.1; 424/130.1; 424/141.1; 424/94.1; 514/7.4; 514/9.6

(57) ABSTRACT

Reversible gel protein formulations, including pharmaceutical formulations, that inhibit protein aggregate formation are described, along with containers, e.g., a vial, ampoule or bottle; or an apparatus or device, e.g., a syringe, comprising such formulations. Also described are methods for inhibiting protein aggregation in a protein-containing sample and methods for inhibiting particle formation in a protein-containing sample.

### RELATED APPLICATIONS

This application claims the benefit of U.S. Provisional Application No. 61/313,044, filed Mar. 11, 2010 which is hereby incorporated by reference.

### FIELD OF THE INVENTION

The invention relates to protein formulations that inhibit insoluble protein aggregate formation.

### BACKGROUND

In the biopharmaceutical industry, protein drug products are subjected to a number of stresses during manufacturing (e.g., purification, formulation and fill & Finish), transportation and storage. These and other stresses can make such protein drug products, e.g., enzymes, antibodies, peptides, and other protein fragments, susceptible to the formation of soluble or insoluble particles or aggregates, both in aqueous solution and when stored frozen.

Although freezing may offer some'protection from degradation (e.g., by slowing the kinetics of various degradation processes), some proteins formulations are not amenable to freezing. Further, it is clear that some protein degradation can occur in the frozen state (e.g., Chang, et al., J. Pharm. Sci. 1996; 85(12):1325-1330; Carpenter and Crow, Cryobiology, 1988; 25:244-255).

Protein molecules may aggregate by physical association with one another without any changes in primary structure (physical aggregation) or by formation of a new covalent bond(s) (chemical aggregation). Formation of such a bond(s) can either directly crosslink proteins (aggregation), or indirectly alter the aggregation tendency of the original protein (Finke et al., 2000, Biochemistry 39, 575-583). Both mechanisms can occur simultaneously to a protein and may lead to formation of either soluble or insoluble aggregates, depending on the protein, environmental condition, and stage of the aggregation process (Wang, W., International Journal of Pharmaceutics 289 (2005) 1-30). Sometimes, initial protein aggregates are soluble but gradually become insoluble as they exceed certain size and solubility limits (Fink, 1998, Fold Des. 3, R9-R23; Uversky et al., 1999, Protein Sci. 8, 161-1). The formation of insoluble aggregates is usually irreversible, unless precipitation is artificially induced such as during salting out (Wang, W., 2005, supra).

The suppression of insoluble aggregate formation during the development, formulation, shipping and storage of a therapeutic protein product is important for maintaining consistency and, in some cases, biological activity of the drug substance, because insoluble aggregate formation can lead to unusable protein material. Numerous processes and additives are known for the stabilization of proteins in solution. For example the stabilization of proteins by adding heat-shock proteins such as HSP25 is described in EP-A 0599344. Antibody stabilization by addition of block polymers composed of polyoxypropylene and polyoxyethylene in combination with phospholipids is described in EP-A 0318081. Immunoglobulins have been stabilized by adding a salt of a nitrogen-containing base, such as arginine, guanidine, or imidazole. Other suitable additives for stabilization are polyethers (EP-A 0018609), glycerin, albumin and dextran sulfate (U.S. Pat. No. 4,808,705), detergents and surfactants such polysorbate-based surfactants (DE 2652636, GB 8514349), chaperones such as GroEL (Mendoza, J. A., Biotechnol. Tech., (10)1991 535-540), citrate buffer (WO 93/22335) or chelating agents (WO 91/15509). Although these additives enable proteins to be stabilized to some degree in solution, they suffer from certain disadvantages, for example, the necessity of additional processing steps for additive removal.

Freeze drying (lyophilization) is considered useful and effective for preservation of many biologically active materials, including proteins (U.S. Pat. No. 6,020,469). However, lyophilization induces its own stresses, including extreme concentration of the protein during the freezing process and removal of water, which may result in instability of the product. Hence, lyophilization may result in increased rates of crosslinking (covalent oligomer formation) and noncovalent aggregation, in addition to deamidation and oxidation, both of which can occur in the lyophilized state as well as the liquid state.

Thus, there remains a need in the art for protein formulations that preferably remain in an unfrozen, non-lyophilized state but have increased stability.

### SUMMARY

In one aspect, the invention includes a pharmaceutical formulation, e.g., a protein formulation, comprising a therapeutic protein and a substance, e.g., a gel, which enables the formulation to exist in a gel phase during storage or transport, and in a liquid phase when administered to a patient. In one embodiment, transition from the gel phase to the liquid phase is achieved by a change in the pH of the formulation. In another embodiment, transition from the gel phase to the liquid phase is achieved by a change in the salt concentration of the formulation. In another embodiment, transition from the gel phase to the liquid phase is achieved by a change in the temperature of the formulation. In one embodiment, the formulation exists in a gel phase below a transition temperature and in a liquid phase above the transition temperature.

In another aspect, the invention includes a pharmaceutical formulation, e.g., a protein formulation, comprising a therapeutic protein and a composition capable of causing the formulation to gel, wherein the formulation is in a gel phase below a transition temperature and in a liquid phase above the transition temperature. In one embodiment, the transition temperature is between about 0° C. and 37° C. In, another embodiment, the transition temperature is between about 4° C. and about 35° C. In another embodiment, the transition temperature is between about 8° C. and about 30° C. In one embodiment, the composition capable of causing the formulation to gel is a gel. In one embodiment, the gel is a hydrogel. In one embodiment, the gel is a thermoreversible or thermosensitive gel. In one embodiment, the gel is a thermoreversible or thermosensitive hydrogel.

In one embodiment, the hydrogel is a gelatin. In a related embodiment, the gelatin is a non-hydrolyzed gelatin. In another embodiment, the gel is a carrageenan. In another embodiment, the gel is agar. In another embodiment, the get is a pectin, e.g., amylopectin. In another embodiment, the gel is a polysaccharide. In another embodiment, the gel is a cellulose derivative, e.g., a methyl cellulose. In another embodiment, the gel is selected from the group consisting of a PEG/PPO block copolymer, a PAA-g poloxamer, a PEG-PLLA-PEG triblock copolymer, a poly (vinyl alcohol) hydrogel, a PEG-PLGA-PEG block copolymer, a poloxamer poly(acrylic acid), a poloxamer-g-PAA hydrogels, a poly(vinyl) alcohol (PVA) hydrogel.

In one aspect, the invention includes a protein formulation comprising a therapeutic protein and gelatin, wherein the formulation exists in a gel phase below about 4° C. and in a liquid phase above about 35° C.

In any of the above aspects or embodiments, the therapeutic protein may be an antibody or fragment thereof. For example, the antibody or fragment thereof may be (or may be derived from) a monoclonal antibody; may be selected from the group consisting of an IgG1, IgG2, IgG3 and IgG4 antibody; may be selected from the group consisting of a fully human antibody, a humanized antibody and a chimeric antibody; or may be selected from the group consisting of a Fab fragment, a Fab′ fragment, a F(ab′)₂ fragment, a single-chain antibody, and a domain antibody.

In any of the above aspects or embodiments, the therapeutic protein may be a peptide or polypeptide; it may be a recombinant form of a native protein such as a receptor, ligand, enzyme or cytokine; and/or it may be a monomer domain binding protein based, on a domain selected from the group consisting of LDL receptor A-domain, thrombospondin domain, thyroglobulin domain, trefoil/PD domain, EGF domain, Anato domain, Notch/LNR domain, DSL domain, Anato domain, integrin beta domain, and Ca-EGF domain.

In one aspect, the invention provides a protein formulation having increased stability against insoluble aggregate or particulate formation induced by physical shock or stress. In one embodiment, the physical stress is stress due to jarring or jostling, such as may occur during shipping and handling of the protein formulation. In, another embodiment, the stress is heat stress, chemical stress (e.g., pH, low/high salt, and the like), fluid stress (e.g., compression stresses, such as those caused by fluid movement through constricted openings). In one embodiment, such a formulation comprises a protein or protein fragment; e.g., a therapeutic protein, and a substance which causes the formulation to exist as a gel below a transition temperature, and as a liquid above the transition temperature, e.g., where the transition temperature is greater than 4° C. and less than 35° C.

In another aspect, the invention provides a protein formulation having increased stability during long-term storage at a refrigerated temperatures, e.g., at a temperature between about 2° C. and 6° C., e.g., storage for a period of 6 months, 8. months, 10 months, 12 months or longer. In one embodiment, such a formulation comprises a protein or protein fragment; e.g., a therapeutic protein, and a substance capable of existing as a gel below a transition temperature, and as a liquid above the transition temperature.

In one embodiment, the pharmaceutical formulation contains, in addition to a gel and a therapeutic protein, one or more of the following: a pharmaceutically acceptable diluent, buffer system, carrier, solubilizer, emulsifier, preservative and/or other component as described below.

In another aspect, the invention includes a container, e.g., a vial, ampoule or bottle; an apparatus or device, e.g., a pre-filled syringe; wherein the contained, apparatus or device contains a pharmaceutical or protein formulation as described above.

In another aspect, the invention includes methods for inhibiting protein aggregation in a liquid protein-containing sample, comprising introducing to the sample a substance capable of causing the sample to gel below a transition temperature, where the transition temperature is greater than 2° C. and less than 35° C.

In another aspect, the invention includes methods for inhibiting particle formation in a liquid protein-containing sample, comprising introducing to the sample a substance capable of causing the sample to gel below a transition temperature, where the transition temperature is greater than 2° C. and legs than 35° C.

In another aspect, the invention includes methods for making pharmaceutical or protein formulations as described above, including methods described herein.

### BRIEF DESCRIPTION OF THE DRAWINGS

FIG. 1 shows an overview of the design of certain experiments with mAb1 performed in support of the present invention.

FIG. 2 shows a photograph of a liquid (left vial) and a thermoreversible gel formulation (right vial) of mAb1 (60 mg/ml mAb1 in A52S).

FIG. 3 shows examples of reduced visible particles in photographs of thermoreversible gel formulations of mAb1 as compared with liquid formulations of mAb1.

FIG. 4 is a graph showing results of sub-visible particle counting.(2 μm-50 μm) analyses obtained using an HIAC instrument.

FIG. 5 is a graph showing results of sub-visible particle counting (2 μm-50 μm) analyses obtained using a MFI instrument.

FIG. 6 shows the results of a visible particle analysis (in the form of photographs) from an 8-month incubation at 4-6° C. (stability study) with liquid and gel formulations of mAb1.

FIG. 7 shows the results of a sub-visible particle analysis by HIAC and MFI following stirring stress of gel and liquid formulations of mAb2

### DETAILED DESCRIPTION

The section headings used herein are for organizational purposes only and are not to be construed as limiting the subject matter described. Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

### Definitions

The term “thermosensitive” or “thermoreversible” substance or composition, such as a gel, e.g., a hydrogel, refers to a substance or composition that can exist in a gel state or phase, as well as in a liquid state or phase, depending on the temperature of the substance.

The term “transition temperature” as used herein refers to the temperature above which a particular thermosensitive or thermoreversible substance or composition is in the liquid phase, and below which the thermosensitive or thermoreversible substance or composition is in the gel phase.

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified (e.g., by the addition of carbohydrate residues to form glycoproteins) or phosphorylated. Polypeptides and proteins can be produced by a naturally-occurring or non-recombinant cell and/or by a genetically-engineered or recombinant cell. Polypeptides and proteins may comprise molecules having the amino acid sequence of the native protein, or molecules having deletions from, additions to, and/or substitutions of one or more ammo acids of the native sequence. Polypeptides and proteins may also be designed de novo or selected from libraries (and optionally further optimized) to bind to selected targets. Examples of targets include human receptors, ligands, hormones, enzymes and cytokines. Examples of target-binding peptides & polypeptides include domain-based polypeptides, including monomer domain polypeptides based on or derived from LDL receptor A-domains, thrombospondin domains, thyroglobulin domains, trefoil/PD domains, EGF domains, Anato domains, Notch/LNR domains, a DSL domains, Anato domains, integrin beta domain, or Ca-EGF domains (see, e.g., US Patent App Pub No. 2006 223114 and US Patent App Pub No. 2006 234299). Examples of polypeptides and proteins include antibodies and fragments thereof, domain-based proteins, target-binding peptides and polypeptides, native proteins and peptides, and modifications of any of the foregoing.

The term “protein aggregate” or “protein aggregation” as used herein refers to a protein that has precipitated and is thus insoluble, or no longer “in solution”. Protein aggregates, as that term is used in the art, can be soluble or insoluble; however for the purposes of the present invention and specification, protein aggregates are considered to be insoluble, unless otherwise specifically noted. Insoluble aggregates whose formation can be reduced or prevented by the compositions, methods and processes according to the invention are understood to refer to protein aggregates typically having a size of at least about 1 μm and can range up to about 2, 5, 10, 15, 20, 25, 50 and 100 μm, or more. The particles can be determined or quantitated by suitable particle counting methods using commercial particle counting instruments such as, for example, the methods described in Example 1 below, and/or other methodologies, e.g., the particle counting instrument AccuSizer 700 from PSS (Particle Sizing. Systems, USA) or a Pacific Scientific HIAC Royco liquid particle counting system, model 9703, equipped with a LD400 laser counter. According to the USP (US-Pharmacopoeia) a maximum of 6000 particles in the range above 10 μm and a maximum of 600 particles in the range above 25 μm are allowed per injected dose of a pharmaceutical preparation.

The term “antibody” refers to an intact immunoglobulin of any isotype, or an antigen binding fragment thereof that can complete with the intact antibody for specific binding to the target antigen, and includes, e.g., chimeric, humanized, fully human, and bispecific antibodies. An “antibody” as such is a species of an antigen binding protein. An intact antibody generally will comprise two full-length heavy chains and two full-length light chains, but in some instances may include fewer chains such as antibodies naturally occurring in camelids which may comprise only heavy chains. Antibodies may be derived solely from a single source, or may be “chimeric,” that is, different portions of the antibody may be derived from two different antibodies as described further below. The antigen binding proteins, antibodies, or binding fragments may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and mutations thereof, e.g., antigen binding fragments, Fab fragments, Fc regions, Fab′ fragments, F(ab′)₂ fragments, Fv regions, single-chain antibodies, and domain antibodies.

The term “light chain” includes a full-length light chain and fragments thereof having sufficient variable region sequence to confer binding specificity. A full-length light chain includes a variable region domain, V_{L}, and a constant region. Domain, C_{L}. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains may be of any isotype, including IgG (including IgG1, IgG2, IgG3 and IgG4 subtypes), IgA (including IgA1 and IgA2 subtypes), IgM and IgE.

The term “antigen binding fragment” (or simply “fragment”) of an antibody or immunoglobulin chain (heavy or light chain), as used herein, comprises a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen binding proteins, including intact antibodies. Immunologically functional immunoglobulin fragments include, but are not limited to, Fab, Fab′, F(ab′)₂, Fv, domain antibodies and single-chain antibodies, and may be derived from any mammalian source, including but not limited to human, mouse, rat, camelid or rabbit.

An “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

An “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. No. 4,946,778 and No. 5,260,203, the disclosures of which are incorporated by reference.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

### Insoluble Aggregate Formation Induced by Physical Shock

One source of protein aggregation is agitation and physical shock, induced, for example, by undue jostling and rough handling of drug product containers during transport or shipping. During shipping, proteins may also interact with hydrophobic surfaces on a glass container or a plastic syringe as well as micro air bubbles in solution or air surface in a container. Such interactions of proteins with hydrophobic materials can also induce protein aggregation. Experiments performed in support of the present invention indicate that protein aggregation induced by physical shock, such as shock during processing, manufacturing, shipping, and storage, results in the formation of insoluble aggregates.

### Gel-Sol Formulations

Experiments performed in support of the present invention are consistent with gel-phase formulations having decreased molecule diffusion-collision events relative to liquid phase formulations, thus diminishing the probability for protein self-association leading to particulation. The results support suppressed particulation (as well as overall increase in protein stability) in gel-phase formulations regardless of the nature of the therapeutic protein and any additional excipient(s) and/or compositions. The results further suggest that the impact of container leachables and extractables is substantially reduced due the diminished diffusion and the physical barrier provided by a gel-phase formulation. For instance, due to its hydrophobicity and limited diffusion, it is believed that silicon oil would be substantially precluded from penetrating into the gel phase and promoting protein self-association/particulation in the gel formulation.

### Gels

In certain concentrations, dispersions of colloids tend to form networks resulting in solid masses, particularly when the solubility of the colloidal material is reduced by change in temperature. These jelly-like semi-solids are known as gels. By weight, gels are mostly liquid, yet they behave like solids due to a three-dimensional crosslinked network within the liquid.

A “hydrogel” is a network of polymer chains that are water-insoluble, sometimes found as a colloidal gel in which water is the dispersion medium. Hydrogels are typically composed of hydrophilic homopolymer or copolymer networks and can swell in the presence of water or physiological liquids (Klouda, L., and Mikos, A. G. (2007), European J. Pharm. Biopharm. 68, 34-45). Thermosensitive or thermoreversible hydrogels, which can exist as either an aqueous polymer solution liquid phase or a gel phase, depending on the temperature, can have applications in biomedical and pharmaceutical applications, such as drug delivery formulations.

A number of studies and applications have been described where the formulation comprises a hydrogel having reverse thermal gelation properties, in which aqueous solutions are liquid at or below ambient temperature and form gel at higher (i.e., body) temperature (Schmolka, I. R., J. Am. Oil Chem. Soc. 1991 68:206-209; Jeong, B., Kim, S. W., and Bae, Y. H. (2002) Thermosensitive sol-gel reversible hydrogels. Adv. Drug Delivery Reviews. 54, 37-51)). For example, Kim and Jin (WO 2009 028764) describe a combination gel of a complex coacervate and a thereto-reversible polysaccharide useful for protein drug delivery, where the gel is water-like sol form at room temperature to facilitate injection, but rapidly forms a gel upon injection into the body to allow for slow and sustained release of the protein contained in the gel. Tonon and Morpugro (WO 2007 115860) describe the use of wet silica based polymers obtained by the sol-gel technology for the entrapment and sustained release of macromolecular bioactive compounds. The wet-gel formulations may then be used for the manufacture of an injectable solution by mixing the silica wet-gel formulation with water or with a physiological water solution. Cha, et al., (WO 1997 15287) describe a biodegradable block copolymer drug delivery system with reverse thermal gelation. The liquid is an aqueous solution containing a drug in a biodegradable block copolymer matrix, where the copolymer has a reverse gelation temperature below the body temperature of the animal to which it is administered.

Hydrogels may be formed from various starting materials, including peptides and proteins. For example peptides can be designed to reversibly adopt well-defined beta-hairpin/sheet or helical secondary structures that can be utilized as topologically defined building blocks (Rajagopal, K., and Schneider, J. P. (2004) Curr. Opin. Struct. Biol. 14, 480-486).

Gelatin is an exemplary hydrogel suitable for use with the present invention. Gelatin is a protein that can be produced, e.g., by partial hydrolysis of collagen extracted from the bones, connective tissues, organs and some intestines of animals, such as domesticated cattle, pigs, and horses. Gelatin, including pharmaceutical grade gelatin, is commercially-available from a number of vendors. Formulations containing suitable concentrations of gelatin (e.g., from about 0.1% to about 10%; from about 0.2% to about 2%) have favorable gel-sol transition temperatures (below body temperature, e.g., ˜37° C. and above refrigeration temperature, e.g., ˜4° C.). Gelatin is included in the US Food and Drug Administration's (FDA's) listing of substances generally regarded as safe (“GRAS”; see the Alphabetical List of SCOGS Substances on the FDA's website); hydrolyzed gelatin (which does not from a gel because its gelling functionality has been enzymatically removed) has been approved by regulatory agencies for use in injectable, pharmaceutical formulations, including vaccines (see, e.g., the FDA's web site; or Liska, V., et al., Journal of Immune Based Therapies and Vaccines 2007, 5:4). Additional references addressing gelatin include de Carvalho, W., et al., (1997) Rheol. Acta 36:591-609; von Hippel, P. H., et al., (1963) Biochemistry 2:1399-1413; and Gelatine Handbook: Theory and Industrial Practice by R. Schrieber and H. Gareis, Wiley Interscience 2007, ISBN: 978-3-527-31548-2.

Other polymers having thermoreversible gelation may also be employed, including polysaccharides and amylopectin cellulose derivatives, many of which have been approved as injectable excipients. Suitable biopolymers preferably form gels in water rather than in organic solvents and form a gel phase on lowering the temperature. For example in the case of polysaccharides, random coils are believed to form helices which subsequently aggregate to form the junction zones of a gel at lower temperature. Artificial polymers such as certain PEG/PPO block copolymers exhibit sol to gel and gel to sol phase temperatures and have been widely studied. PAA-g Poloxamers as well as PEG-PLLA-PEG triblock copolymers of specific molecular weight are also believed to have utility in this regard. Poly (vinyl alcohol) hydrogels that exist in gel state below room temperature formed as a consequence of crystallization of PVA molecules may also be utilized.

The preparation and properties of a number of polymer-based hydrogels suitable for use with the present invention are described in the literature. They include natural polymers such as cellulose derivatives, e.g., methyl cellulose of various molecular weights where gelation is promoted by increase in molecular weight of MC (Nishinari, K., Hofmann, K. E., Moritaka, H., Kohyama, K., and Nishinari, N. (1997) Gel-sol transition of methylcellulose. Macromolecular Chemistry and Physics 198, 1217-1226.). Also described are PEG-PLGA-PEG block copolymers (Jeong, B., Kim, S. W., and Bae, Y. H. (2002) Thermosensitive sol-gel reversible hydrogels. Adv Drug delivery Reviews 54, 37-51; Peppas, N. A., Hilt, J. Z., Khademhosseini, A., and Langer, R. (2006) Hydrogels in biology and medicine: From molecular principles to Bionanoteehnology. Adv. Mater. 8, 1345-1360), which are known for thermogelation, biodegradability and lack of toxicity, and are proposed for injectable gel systems. Changing the polymer composition, mainly the middle block composition, blocks length and block ratio can be used to modulate the temperature of gel-sol transition. Additional suitable gels include Poloxamer poly(acrylic acid) or Poloxamer-g-PAA hydrogels (Jeong, B., et al., Adv Drug delivery Reviews 54:37-51; Peppas, N. A., et al., Adv. Mater. 18:1345-1360) that exhibit gelation in water and can be formed by coupling poloxamer to FAA using cross-linking agent that can be removed through extraction process. These hydrogels contain pH-sensitive functional groups and their gelation property can be also modulated by salt. Also suitable are Poly(vinyl) alcohol (PVA) hydrogels, which have high mechanical strength and excellent transparency (Ryon, S. et al., (1989) Polymer Bulletin 22:119-122) as well as being biodegradable; however, the cross-linking agent residue preferably needs to be removed by extraction procedures to render formulations using PVA hydrogels suitable for therapeutic application.

### Reversible Gel-Liquid Formulation

Methods and compositions of the present invention are useful with a broad variety of proteins, buffers, excipients, containers (e.g., pre-filled syringes), and the like. For example, gel-based protein formulation as described herein may be packaged in vials, syringes or larger containers, and may be shelved, stored or transported, e.g., at between about 0° C. and 8° C. or between about 2° C. and 6° C., or at about 4° C., with the formulation in the gel phase. Prior to administration, the formulation (or container in which the formulation is contained) is heated, e.g., to between about 25° C. and 35° C., transforming the formulation from a gel phase to a liquid phase, which can be utilized for drug delivery, e.g., via injection. In the case of orally-available therapeutic molecules, the formulation may be ingested as a gel, dissolving as it is consumed & passed down the digestive tract. Similarly, in the case of therapeutics applied via routes of administration other than orally or via injection, e.g., nasally, rectally or virginally, the formulation may be applied as a gel and allowed to transform into a liquid state as it warms up to and is maintained at body temperature.

### Pharmaceutical Formulations

Pharmaceutical formulations, e.g., protein-containing formulations, as described herein employ gels, e.g., hydrogels, having standard gelation properties, where the formulation or composition is in a gel state or form at or below ambient temperature and in a liquid form at higher, “body”, temperature.

In one aspect, pharmaceutical formulations (e.g., protein formulations) of the present invention comprise a therapeutic protein and a substance, e.g., a gel, which causes the formulation to exist in a gel state or phase below the transition temperature. Acceptable formulation materials preferably are nontoxic to recipients at the dosages and concentrations employed. In certain embodiments, the pharmaceutical composition may additionally contain formulation materials for modifying, maintaining or preserving, for example, the pH, osmolarity, viscosity, clarity, color, isotonicity, odor, sterility, stability, rate of dissolution or release, adsorption or penetration of the composition. In such, embodiments, suitable additional formulation materials include, but are not limited to, amino acids (such as glycine, glutamine, asparagine, histidine, arginine or lysine); antimicrobials;, antioxidants (such as ascorbic acid, sodium sulfite or sodium hydrogen-sulfite); buffers (such as borate, bicarbonate, Tris-HCl, citrates, phosphates, acetates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); monosaccharides; disaccharides; and other carbohydrates (such as glucose, mannose or dextrins); proteins (such as serum albumin or immunoglobulins); coloring, flavoring and diluting agents; emulsifying agents; low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or Sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo; ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in viva release and rate of in vivo clearance of the therapeutic protein. In certain embodiments, the primary vehicle or carrier in a pharmaceutical composition, which in all cases contains a substance, e.g., a gel, capable of causing the pharmaceutical composition to exist in a gel state or phase below the transition temperature, may be either aqueous or non-aqueous in nature. For example, a suitable vehicle or carrier may be water for injection, physiological saline solution or artificial cerebrospinal fluid, possibly supplemented with other materials common in compositions for parenteral administration; neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5; or phosphate buffer, and may further include sorbitol or a suitable substitute. In certain embodiments, therapeutic protein compositions may be prepared for storage by mixing the selected composition having the desired degree of purity with optional formulation agents (REMINGTON'S PHARMACEUTICAL SCIENCES, supra) in the form of an aqueous solution which further contains a substance, e.g., a gel, capable of enabling the solution to exist in a gel state or phase below the transition temperature.

The pharmaceutical compositions can be selected for parenteral delivery. Alternatively, the compositions may be selected for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers may be used to maintain the composition at physiological pH or at a slightly lower pH, for example, within a pH range of from about 5 to about 8. Suitable pH ranges for the preparation of the formulations will depend on the particular protein or protein fragment of interest. It is particularly advantageous to select a buffer with a pH range that retains its buffering capacity in a range greater than or equal to 1 pH unit larger or smaller than the isoelectric point (pI) of the protein of interest. In many cases, the pH of the buffer system is stable in a range greater than or equal to 2 pH units larger or smaller than the pI of the protein. Further, it may be advantageous to select a buffer system that maintains pH over a large range of temperatures. That is, the pH of the buffer system is preferably not significantly temperature dependent or responsive. In one embodiment the buffer is a potassium phosphate/potassium acetate mixed buffer system, having a pH range of about 4 to about 8, and a concentration range of about 1 mM to about 300 mM.

When parenteral administration is contemplated, the therapeutic compositions may be provided in the form of a pyrogen-free, parenterally acceptable aqueous solution comprising the desired therapeutic protein in a pharmaceutically acceptable vehicle comprising a substance, e.g., a gel, capable of enabling the solution to exist in a gel state or phase below the transition temperature. A particularly suitable vehicle for parenteral injection is sterile distilled water in which the therapeutic protein is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads, or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Some formulations can be administered orally. Therapeutic proteins that are administered in this fashion can be formulated with or without carriers customarily used in the compounding of solid dosage forms such as capsules. In certain embodiments, a capsule may be designed to release the active portion of the formulation at the point in the gastrointestinal tract when bioavailability is maximized and pre-systemic degradation is minimized. Additional agents can be included to facilitate absorption of the therapeutic protein.

Pharmaceutical compositions used for in vivo administration are typically provided as sterile preparations. Sterilization can be accomplished by filtration through sterile filtration membranes at a temperature where the pharmaceutical composition is in a liquid state. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials below the transition temperature as a gelled solution or suspension. Kits for producing a single-dose administration unit are also provided. In certain embodiments, kits containing single and multi-chambered pre-filled syringes (e.g., liquid syringes and lyosyringes) are provided. The therapeutically effective amount of a therapeutic protein-containing pharmaceutical composition to be employed will depend, for example, upon the therapeutic context and objectives. One skilled in the art will appreciate that the appropriate dosage levels for treatment will vary depending, in part, upon the molecule delivered, the indication for which the therapeutic protein is being used, the route of administration, and the size (body weight, body surface or organ size) and/or condition (the age and general health) of the patient. In certain embodiments, the clinician may titer the dosage and modify the route of administration to obtain the optimal therapeutic, effect.

The route of administration of the pharmaceutical formulation is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The following examples, including the experiments conducted and the results achieved, are provided for illustrative purposes only and are not to be construed as limiting the scope of the appended claims.

### Example 1

### Drop-Shock Induced Aggregation

Samples of two different monoclonal antibodies were prepared in standard liquid formulations, as well as in gel formulations (using gelatin) according to an aspect of the present invention. The formulations were subjected to drop shock, which can induce protein aggregation or particulation in liquid formulations of certain proteins.

### A. Methods

1. Sample Preparation

Gelatin from porcine skin (electrophoresis grade, Type A) was obtained from Sigma-Aldrich (St. Louis, Mo.). Purified human IgG monoclonal antibodies (mAbs) were formulated in acetate buffer with or without gelatin. The gelatin (from porcine skin; electrophoresis grade, Type A) was obtained from Sigma-Aldrich (St. Louis, Mo.). Antibody mAb1 was prepared as a 60 mg/ml solution in 10 mM sodium acetate buffer, pH 5.2 containing 5% (Aviv) sorbitol (“A52S”), with or without 0.5% gelatin. Antibody mAb2 was prepared as a 1 mg/ml solution in 10 mM Acetate pH 5.0 with or without 1% gelatin, in 5 cc vials. All samples (including gelatin-free controls) were incubated overnight at 4° C. in order to achieve sol-gel transition (i.e., gel state or phase) of gelatin-containing preparations.

2. Light Obscuration (HIAC)

Particle counting and size distribution was achieved via the light obscuration method described in the United States Pharmacopeia (“USP”) General Chapter <788> and adapted for a smaller sample volume, using a HIAC/Royco Liquid Particle Counter Model 9703 with a HRLD-150 sensor and PharmSpec software (Hach Company, Loveland, Colo.). Particle-free water was washed through the system before each sample analysis to provide for a clean baseline. Samples were diluted 20× in A52S buffer (mAb1) or water (mAb2) and placed in 3 cc vials (2.2 ml per vial) to keep the particle count within the detection limits of the instrument. Samples were degassed in a vacuum chamber at 75 torr for 2 hours, gently swirled, and then measured 4 times at a volume of 0.5 ml per injection. The first run was discarded and the last three runs were averaged. Sub-visible particles greater than or equal to 2, 5, 10, 15, 20, 25, 50 and 100 μm were recorded as differential counts per 0.5 ml. Final particle counts were adjusted based on the initial dilution factor.

3. Micro-Flow Imaging (MFI)

Particle images were captured on a liquid-borne particle Micro-Flow Imaging (MFI) System DPA4100 (Brightwell Technologies, Inc). Particle-free water was flushed through the system before each sample analysis to provide for a clean baseline. Samples were diluted 20× in A52S buffer (mAb1) or water (mAb2) in the same manner as described in the light obscuration section. Samples were degassed in a vacuum chamber at 75 torr for 2 hours, gently swirled and then drawn from a 1 ml pipette tip into the flow-cell using a peristaltic pump. A volume of 0.8 ml was loaded at a time, where the first 0.3 ml were discarded and only the following 0.5 ml were analyzed. 500 images were collected per sample and processed by the system software to extract each particle and its characteristics, including size; shape, transparency and an individual image. Typical particle images from each sample with the longest overall dimension (feret diameter) were used for further comparison.

B. Particulation Propensity of Gel-Containing Formulations of mAb1 in Response to Drop Shock

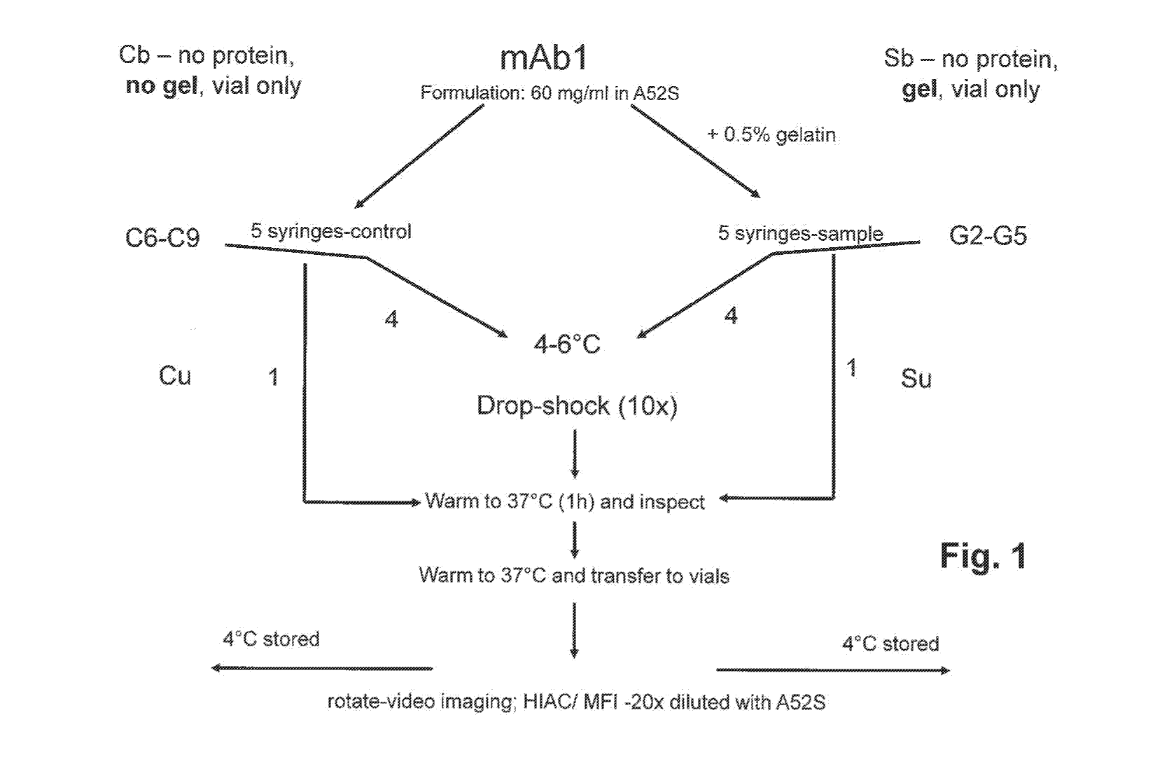

A graphical representation of the experimental protocol used to analyze the particulation propensity or tendency to form aggregates of mAb1 to drop shock in both liquid and gel formulations is shown in FIG. 1.

1. mAb1 Samples

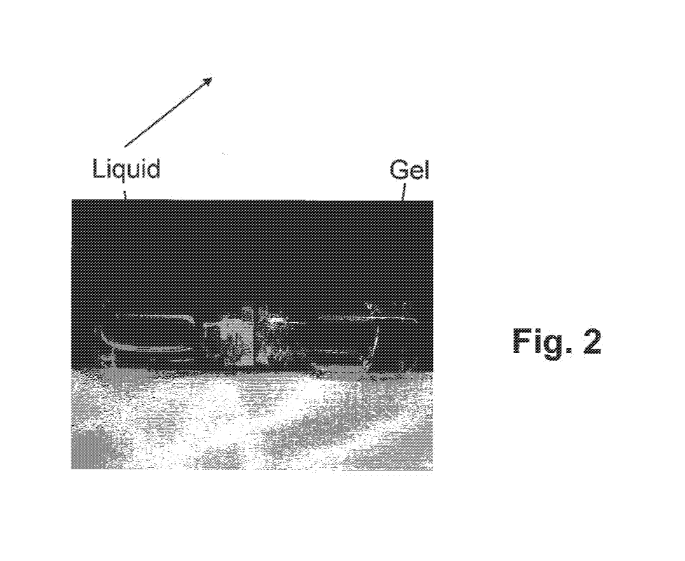

Antibody mAb1 was formulated at 60 mg/ml in A52S buffer with or without gelatin (0.5%), placed in the therapeutic glass syringes and incubated overnight at 4° C., resulting in formation of gel (see FIG. 2). Drop-shock was carried out by dropping the syringe rack to the floor from about a 1.5 m distance ten times at 4° C. Samples were labeled as follows and as illustrated in FIG. 1:

Samples C6, C7, C8, C9, CU and CB were liquid formulations with no gelatin. Samples C6, C7, C8 and C9 were subjected to drop-shock; samples CU and CB were not. Samples C6, C7, C8, C9 and CU contained mAb1 protein; sample CB did not. Samples G2, G3, G4, G5, SU and SB contained 0.5% gelatin. Samples G2, G3, G4, and G5 were subjected to drop-shock, samples SU and SB—were not. Samples G2, G3, G4, G5 and SU contained mAb1 protein; sample SB—did not.

Prior to analyzing the samples for visible and sub-visible particles, all samples (in syringes) were subjected to a one hour incubation at 37° C. to transform or transition the gel phase into a liquid solution. Then, each sample solution was carefully transferred/injected from the syringe into a 3 cc vial. The resulting sample vials were used for qualitative (imaging) and quantitative (HIAC, MFI) particulate analyses.

2. Visible Particle Analyses

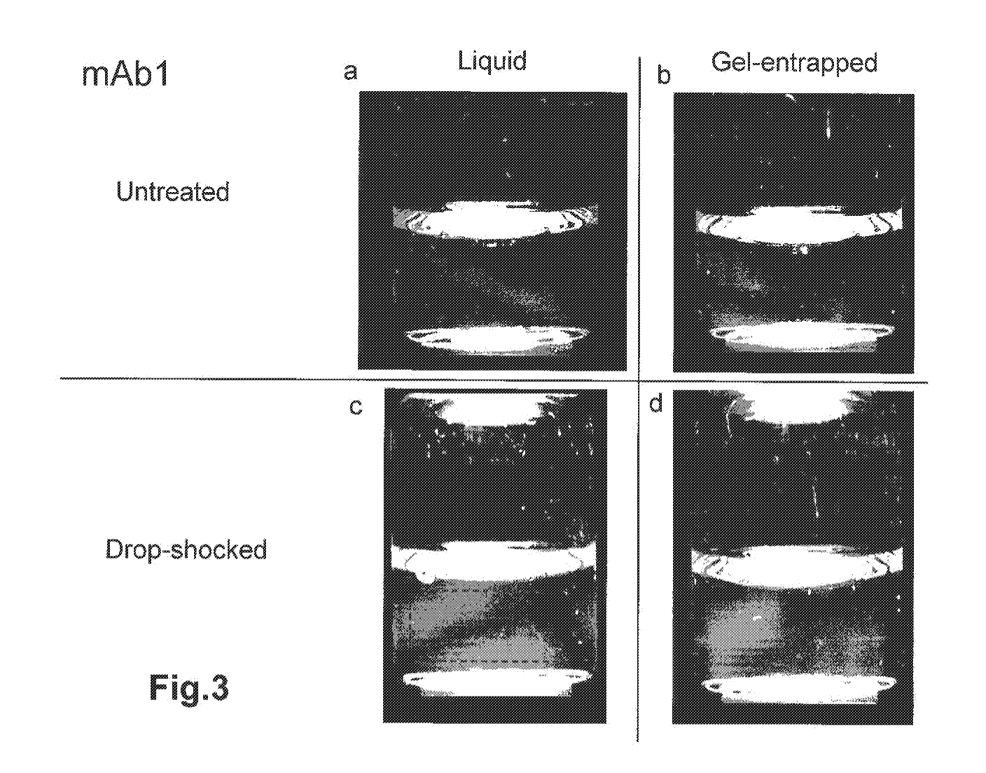

Visual inspection including rotated vial-video imaging analysis showed a dramatic suppression of the shock-induced visible particulation in the gel formulations—whereas many particles were observed in C6-C9 samples (FIG. 3c), few or no particles were observed in gel-containing G2-G5 samples (FIG. 3d). The untreated (no shock) samples were essentially free of visible particles (FIGS. 3a, 3b).

3. Sub-Visible Particle Analyses

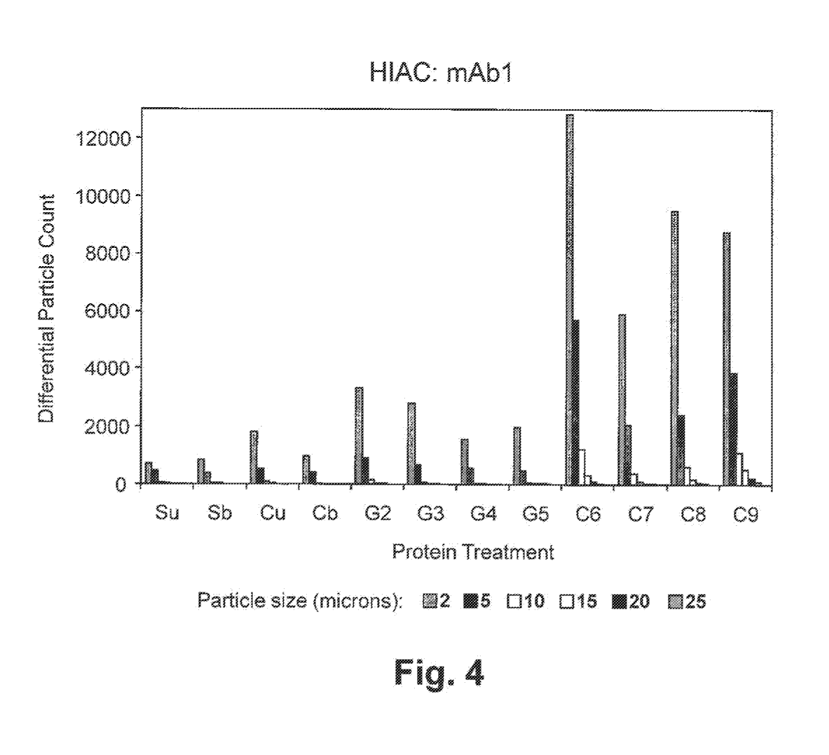

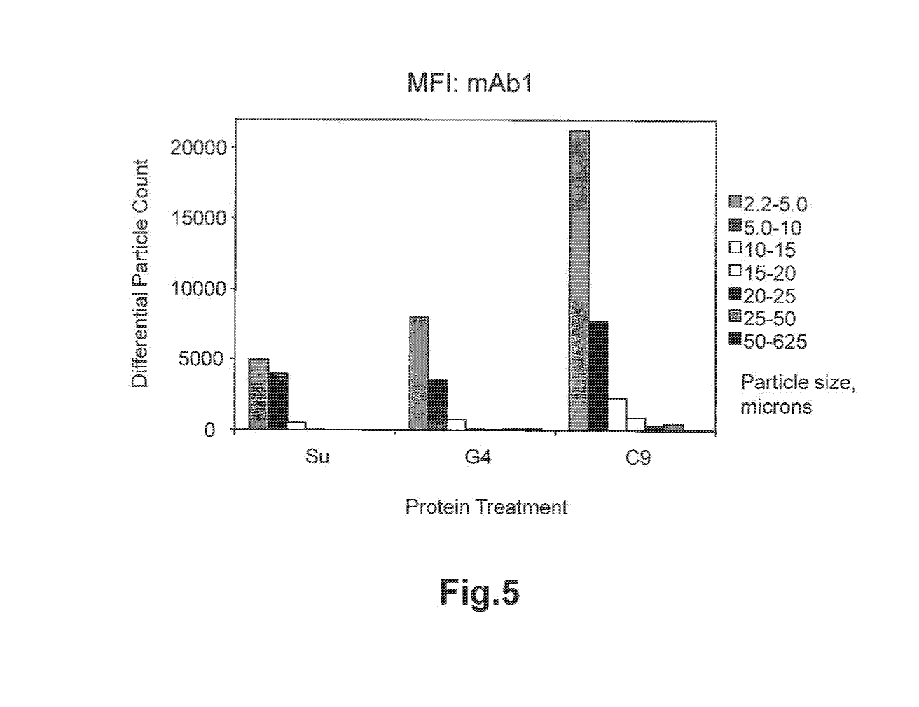

The sub-visible particle counting (2 μm-50 μm) analyses were performed by two different methods using HIAC (FIG. 4) and MFI (FIG. 5) instruments. Results obtained using both methods were consistent with results based on analysis the visible particulation—there was a dramatic suppression of subvisible particulation in the gel-containing G2-G5 samples, which were resistant to shock-induced particulation and showed a subvisible particulation level similar to that of untreated (no shock) material (FIGS. 4 and 5). In contrast, formulations which were not in the gelled state when subjected to the drop shock test showed substantially increased particulation or aggregation propensity.

4. Storage Stability Studies

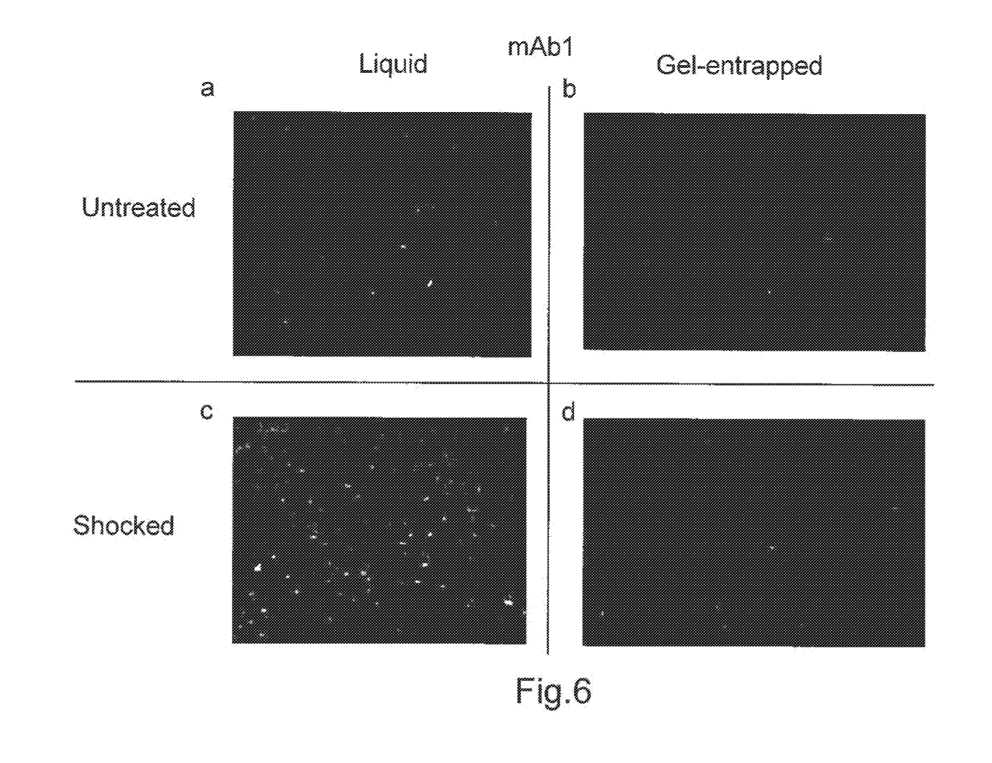

After the particulation analyses, the remaining sample solutions were refrigerated for eight months to a temperature where the gelatin-containing samples were maintained in a gel phase. Following the eight-month period, the samples were heated to 37° C. for 30 minutes and imaged for visible particle analyses as described above. As shown in FIG. 6, more particles were produced during the eight month 4° C. incubation in the non-shocked (untreated) non-gel-containing liquid sample (FIG. 6a) than in the non-shocked (untreated) gel-entrapped sample (FIG. 6b). These results support the position that the gel-entrapped material was (in addition to having improved shock resistance) also more resistant to particulation during storage at 4° C., indicating improved long-term stability. Data generated using the treated (subjected to drop shock) 8-month 4° C. incubated samples (FIGS. 6c, 6d) were consistent with earlier data (FIG. 3c, 3d), showing a dramatic suppression of visible particulation in the gel entrapped protein material as compared with non-gel liquid formulations.

C. Particulation Propensity of Gel-Containing Formulations of mAb2 in Response to Stirring

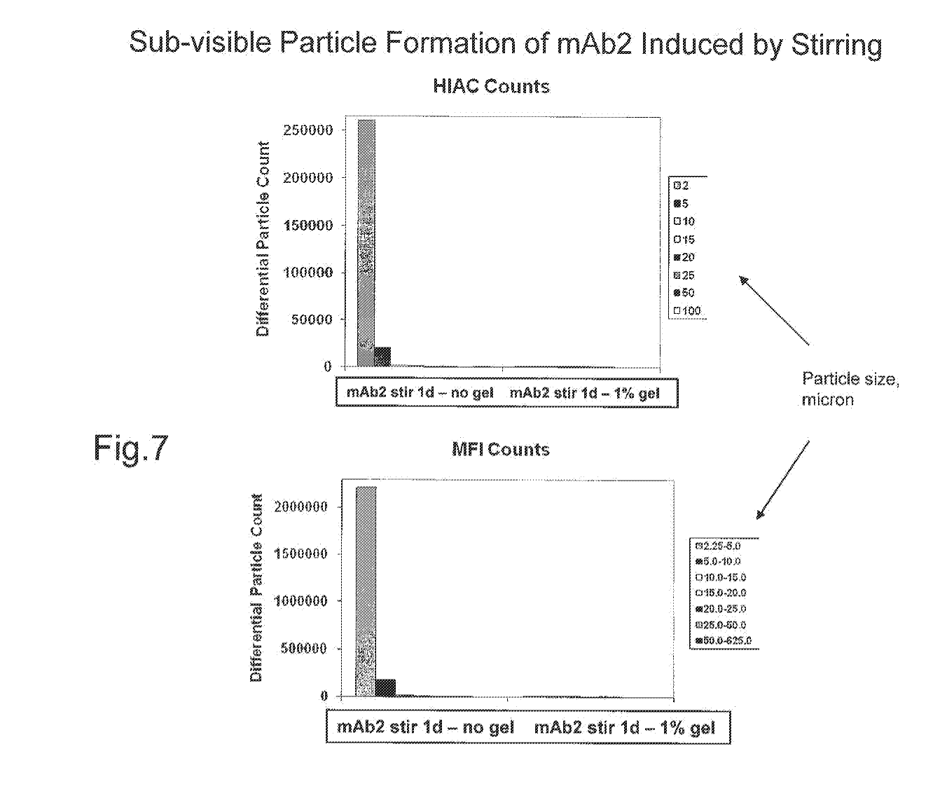

Gel and liquid formulations similar to those described above were prepared using a second monoclonal antibody (mAb2) as the test protein, subjected to a stirring stress at 4° C., and assessed for particulation or protein aggregation propensity. The gel phase was crushed into small pieces prior to the experiments. For stirring stress, the protein samples (with or without gelatin) were stirred for 20 h at 4° C. with a 6 mm×6 mm Teflon stirrer bar at approximately 700 rpm in a glass vial capped and placed vertically on a magnetic stir plate over 20 hours at 4° C. Although no visible particles were generated, the subvisible particle analysis by HIAC and MFI showed almost a total suppression of particle formation in the gel-entrapped material (FIG. 7).

All patents and other publications identified are expressly incorporated herein by reference for the purpose of describing and disclosing, for example, the methodologies described in such publications that might be used in connection with the subject matter disclosed herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

It should be understood that the foregoing disclosure emphasizes certain specific embodiments of the invention and that all modifications or alternatives equivalent thereto are within the spirit and scope of the invention as set forth in the appended claims.

1. A pharmaceutical formulation comprising a therapeutic protein and a gel, wherein said formulation exists in a gel phase below a transition temperature and in a liquid phase above said transition temperature.

2. The composition of claim 1, wherein said transition temperature is between about 0° C. and 37° C.

3. The composition of claim 2, wherein said transition temperature is between about 4° C. and 35° C.

4. The composition of claim 3, wherein said transition temperature is between about 8° C. and 30° C.

5. The composition of claim 1, wherein said gel is a thermoreversible gel.

6. The composition of claim 1, wherein said gel is a hydrogel.

7. The composition of claim 6, wherein said hydrogel is a non-hydrolyzed gelatin.

8. The composition of claim 1, wherein said gel is selected from the group consisting of a carrageenan, a pectin, agar and a polysaccharide.

9. The composition of claim 1, wherein said gel is selected from the group consisting of a PEG/PPO block copolymer, a PAA-g poloxamer, a PEG-PLLA-PEG triblock copolymer, a poly (vinyl alcohol) hydrogel, a PEG-PLGA-PEG block copolymer, a poloxamer poly(acrylic acid), a poloxamer-g-PAA hydrogels, and a poly(vinyl) alcohol (PVA) hydrogel.

10. The composition of claim 1, wherein said therapeutic protein is an antibody or fragment thereof.

11. The composition of claim 10, wherein said antibody or fragment thereof is a monoclonal antibody or fragment thereof.

12. The composition of claim 10, wherein said antibody or fragment thereof is selected from the group consisting of an IgG1 antibody (or fragment thereof), IgG2 antibody (or fragment thereof), IgG3 antibody (or fragment thereof) and IgG4 antibody (or fragment thereof).

13. The composition of claim 10, wherein said antibody or fragment thereof is selected from the group consisting of a fully human antibody (or fragment thereof), a humanized antibody (or fragment thereof) and a chimeric antibody (or fragment thereof).

14. The composition of claim 10, wherein said antibody or fragment thereof is selected from the group consisting of a Fab fragment, a Fab′ fragment, a F(ab′)₂ fragment, a single-chain antibody, and a domain antibody.

15. The composition of claim 1, wherein said therapeutic protein is a peptide or polypeptide.

16. The composition of claim 15, wherein said peptide or polypeptide is selected from the group consisting of a receptor, a soluble receptor, a ligand, an enzyme and a cytokine.

17. The composition of claim 15, wherein said peptide or polypeptide is a monomer or multimer based on one or more domain(s) selected from the group consisting of LDL receptor A-domain, thrombospondin domain, thyroglobulin domain, trefoil/PD domain, EGF domain, Anato domain, Notch/LNR domain, DSL domain, Anato domain, integrin beta domain, and Ca-EGF domain.

18. A protein formulation comprising a therapeutic protein and gelatin, wherein the formulation exists in a gel phase below about 4° C. and in a liquid phase above about 35° C.

19. The protein formulation of claim 18, wherein said formulation has increased stability against insoluble aggregate formation.

20. The protein formulation of claim 19, wherein said insoluble aggregate formation is induced by physical shock or stress

21. The protein formulation of claim 20, wherein said physical shock or stress is stress due to jarring or jostling.

22. The protein formulation of claim 20, wherein said physical shock or stress is selected from the group consisting of heat stress, chemical stress and fluid stress.

23. A pharmaceutical formulation comprising a peptide or protein and a substance which causes the formulation to exist as a gel below a transition temperature, and as a liquid above the transition temperature

24. The formulation of claim 23, wherein the transition temperature is between about 4° C. and about 35° C.

25. The formulation of claim 23, wherein the formulation has increased stability during long-term storage at a temperature between about 2° C. and 6° C.

26. The protein formulation of claim 25, wherein the long-term storage comprises storage for a period of longer than about 6 months.

27. The protein or therapeutic formulation of either of claim 1, wherein said protein or formulation also comprises one or more of the following: a pharmaceutically acceptable diluent, buffer system, carrier, solubilizer, emulsifier and preservative.

28. The protein or therapeutic formulation of claim 15, wherein said protein or formulation also comprises one or more of the following: a pharmaceutically acceptable diluent, buffer system, carrier, solubilizer, emulsifier and preservative.
